(19) United States
(12) Patent Application Publication
(10) Pub. No.: US 2026/0183027 A1
(43) Pub. Date: Jul. 2, 2026

(54) GUIDED DRILL INSERTION FOR SUTURE BUTTONS

(71) Applicant: Acumed LLC, Hillsboro, OR (US)

(72) Inventors: David Ruch, Hillsboro, OR (US); Jerry Huang, Hillsboro, OR (US); Ryan Garcia, Hillsboro, OR (US); Mason Bettenga, Memphis, TN (US); Tristan Sommers, Hillsboro, OR (US); Larry Ehmke, Portland, OR (US); Brandon Wedam, Olympia, WA (US); Manali Paralkar, Tigard, OR (US)

(21) Appl. No.: 19/425,090

(22) Filed: Dec. 18, 2025

Related U.S. Application Data
(60) Provisional application No. 63/740,144, filed on Dec. 30, 2024.

(51) Int. Cl. A61B 17/80; A61B 17/86

(52) U.S. Cl. CPC A61B 17/8057; A61B 17/80; A61B 17/8061; A61B 17/8085; A61B 17/86; A61B 17/8685

(57) ABSTRACT

A system for transporting an anchoring system through at least one bone can include a button leader, a cable, and a lead button. The button leader can include a first leader end and a second leader end and a first mating feature formed on the second leader end. The lead button can include at least one aperture having a cable positioned through the aperture, a leading wing extending from the aperture, and a trailing wing extending from the aperture opposite the leading wing. The leading wing can be operable to mate with the first mating feature. The button leader and the lead button can be configured to be transported together through a channel of at least one bone.

### TECHNICAL FIELD

The present application relates generally to surgical techniques to hold two bones together. More specifically, the present application provides insertion systems and methods that enable surgeons, or any other suitable healthcare provider, to more easily and effectively implement the lead button technique, such as to repair bone injuries.

### BACKGROUND

In various instances, patients may suffer injuries that require securing a first, or near, bone to a second, or far, bone in order to help the patient recover from the injury. Currently, securing implants such as bone plates or intramedullary screws to the bone requires multiple steps and different tools. For example, to secure a bone plate to a bone, surgeons typically first solid drill through the plate hole and out of the far cortex, remove the drill, and insert a screw into the drilled hole. With this method, it can be challenging to find the near cortical hole for insertion of the screw and to accurately insert the screw through the bone and out of the far cortical hole. This method can be even more difficult when there is a second bone plate on the far side of the bone through which the screw needs to traverse.

Another method to secure a near bone to a far bone is the lead button technique. The lead button technique includes deploying a bone-securing construct that includes a first button coupled to a second button with cable. The cable may be tensioned to secure the near bone to the far bone. For example, to repair syndesmotic injuries, the lead button technique involves two buttons that hold the fibula (e.g., near bone) and tibia (e.g., far bone) together with cable that connects the two buttons through a drilled bone hole in the fibula and tibia. The buttons are typically positioned with a needle and pull-through cables or with a button leader. A deployment rod typically can easily access the near bone tunnel, but in some situations, the deployment rod cannot find the far bone tunnel if any shift has occurred, for example from motion between the bones. This problem leads to difficulty in the operating room and frustration if the far bone tunnel cannot be found.

Yet another form of technology is passing the button using pull-through cable to pull the button through the drill hole. One side of the button is attached to a line of suture that is passed through the drill hole using a passing wire. The suture is then used to pull the button through drill hole. If there is any shift in the bones, it may be difficult to get the passing wire through all four cortices. Additionally, there is a risk of the button flipping and becoming anchored between the bones, which is a situation that causes significant additional work to fix and results in surgeon frustration.

Another method is passing only a cable through a drill hole using a k-wire with a passing loop on the back, then attaching the button to the cable on the far bone. A k-wire doesn't maintain hole axial alignment as well as a drill, since the k-wire is so much thinner than a drill. Using the k-wire, the bones could move and cause a challenge for the button being pushed through. If a button is lost at any point, it could get flipped halfway which could cause complications with the procedure. Additionally, the k-wire has less material than a drill, making it difficult to connect to the button as effectively as a drill. Another key disadvantage of this method is that it requires a larger incision over the far bone to access the suture and secure the button with a knot. Additionally, more complicated suture constructs such as knotless suture are difficult to assemble onto buttons in the operating room without sacrificing button integrity.

In carpometacarpal applications, the carpometacarpal (CMC) joint forms the base of the thumb and is where the metacarpal bone of the thumb, also known as the first metacarpal, attaches to the trapezium bone. Cartilage is found at the base of the bones and acts as a cushion, allowing the bones to glide smoothly against each other. Arthritis of the thumb or CMC arthritis is a common problem that occurs when the cartilage wears away from the ends of the bones of the CMC joint. Without cartilage, the thumb metacarpal and the trapezium bone rub directly against each other, which can cause severe pain, swelling, and decreased strength of the thumb.

Treatment for CMC arthritis can include removal of the trapezium bone in a procedure called a trapeziectomy, which creates space and prevents bone on bone interaction between the thumb metacarpal and the trapezium. However, removal of the trapezium can cause other problems. First, the thumb metacarpal can collapse onto the scaphoid, which is called thumb metacarpal subsidence. To remedy this issue, surgeons can implant natural or synthetic material in place of the trapezium. However, the current methods can cause proximal migration of the thumb metacarpal causing the thumb and the index metacarpal to rub together, which is known as impingement.

### SUMMARY

In light of the technical features set forth herein, and without limitation, in a first aspect of the disclosure in the present application, which may be combined with any other aspect unless specified otherwise, a system for transporting an anchoring system through at least one bone includes a button leader, a cable, and a lead button. The button leader includes a first leader end and a second leader end and a first mating feature formed on the second leader end. The lead button includes at least one aperture having a cable positioned through the aperture, a leading wing extending from the aperture, and a trailing wing extending from the aperture opposite the leading wing. The leading wing is operable to mate with the first mating feature. The button leader and the lead button are configured to be transported together through a channel of at least one bone.

In another aspect of the disclosure in the present application, which may be combined with any other aspect unless specified otherwise, a bone drilling feature is formed on the first leader end.

In another aspect of the disclosure in the present application, which may be combined with any other aspect unless specified otherwise, a diameter of the first leader end is greater than a diameter of the second leader end.

In another aspect of the disclosure in the present application, which may be combined with any other aspect unless specified otherwise, a diameter of the first leader end is in a range of about 1.2 mm to about 6.5 mm.

In another aspect of the disclosure in the present application, which may be combined with any other aspect unless specified otherwise, the button leader is cannulated.

In another aspect of the disclosure in the present application, which may be combined with any other aspect unless specified otherwise, the system further includes a button inserter, the button inserter being connected to the lead button, the button inserter further comprises, a second mating feature operable to mate with the trailing wing and a pusher rod. The pusher rod is operable to decouple the lead button from the button inserter.

In another aspect of the disclosure in the present application, which may be combined with any other aspect unless specified otherwise, the system further includes a trigger operable to cause the pusher rod to translate within the button inserter.

In another aspect of the disclosure in the present application, which may be combined with any other aspect unless specified otherwise, the system further includes a fixator that is coupled to the lead button by the cable. The fixator is an anchor or an additional button.

In another aspect of the disclosure in the present application, which may be combined with any other aspect unless specified otherwise, the trailing wing includes a chamfered end.

In another aspect of the disclosure in the present application, which may be combined with any other aspect unless specified otherwise, the chamfered end is chamfered at an angle between 20 to 60 degrees relative to the trailing wing.

In another aspect of the disclosure in the present application, which may be combined with any other aspect unless specified otherwise, the aperture is formed off-center from a midpoint between the leading wing and the trailing wing.

In another aspect of the disclosure in the present application, which may be combined with any other aspect unless specified otherwise, the button inserter further includes a locating window, wherein the position of the locating window with respect to the at least one bone is identifiable on an x-ray.

In another aspect of the disclosure in the present application, which may be combined with any other aspect unless specified otherwise, the at least one bone is selected from a group consisting of a metacarpal, a metatarsal, a tibia, a fibula, an ulna, a radius, and a phalange.

In another aspect of the disclosure in the present application, which may be combined with any other aspect unless specified otherwise, the lead button is passed through channels in multiple bones.

In another aspect of the disclosure in the present application, which may be combined with any other aspect unless specified otherwise, one bone is a first metacarpal and a second bone is a second metacarpal.

In another aspect of the disclosure in the present application, which may be combined with any other aspect unless specified otherwise, the button inserter and the button leader are coupled.

In another aspect of the disclosure in the present application, which may be combined with any other aspect unless specified otherwise, a method of inserting a cable through a first bone and a second bone includes providing a system including a button leader, a button inserter, the cable, and a lead button; drilling a first channel through the first bone using the button leader, wherein the button leader comprises a first leader end having a drilling feature, and a second leader end comprising a first mating feature; drilling a second channel through the second bone using the button leader such that the button leader remains within the first channel; coupling the lead button within the first mating feature, wherein the lead button comprises at least one aperture wherein the cable is positioned through the at least one aperture; pushing the button leader and lead button through the first channel and the second channel via the button inserter such that the at least a portion of the cable is positioned through the first channel and the second channel; and decoupling the lead button from the first mating feature.

In another aspect of the disclosure in the present application, which may be combined with any other aspect unless specified otherwise, the lead button further includes a leading wing extending from the aperture and a trailing wing extending from the aperture. The leading wing is operable to mate with the first mating feature and the trailing wing is operable to mate with a second mating feature, the second mating feature formed in a first end of a button inserter.

In another aspect of the disclosure in the present application, which may be combined with any other aspect unless specified otherwise, the trailing wing includes a chamfered end.

In another aspect of the disclosure in the present application, which may be combined with any other aspect unless specified otherwise, the chamfered end is chamfered at an angle between 20 to 60 degrees relative to the trailing wing.

In another aspect of the disclosure in the present application, which may be combined with any other aspect unless specified otherwise, the aperture is formed off-center from a midpoint between the leading wing and the trailing wing.

In another aspect of the disclosure in the present application, which may be combined with any other aspect unless specified otherwise, the button inserter further comprises a locating window, wherein the position of the locating window with respect to the at least one bone is identifiable on an x-ray.

In another aspect of the disclosure in the present application, which may be combined with any other aspect unless specified otherwise, the button leader comprises a first leader end and a second leader end, a diameter of the second leader end is less than a diameter of the first leader end.

In another aspect of the disclosure in the present application, which may be combined with any other aspect unless specified otherwise, a diameter of the first leader end is greater than a diameter of the button inserter.

In another aspect of the disclosure in the present application, which may be combined with any other aspect unless specified otherwise, a diameter of the first leader end of the button is in a range of about 1.2 mm to about 6.5 mm.

In another aspect of the disclosure in the present application, which may be combined with any other aspect unless specified otherwise, the button leader is cannulated.

In another aspect of the disclosure in the present application, which may be combined with any other aspect unless specified otherwise, the button inserter includes an inserter shaft having the second mating feature formed therein, a pusher rod, the pusher rod is operable to translate within the inserter shaft and cause the button to translate with respect to the inserter shaft.

In another aspect of the disclosure in the present application, which may be combined with any other aspect unless specified otherwise, the button inserter further comprises a trigger, wherein activating the trigger causes the pusher rod to translate within the inserter shaft.

In another aspect of the disclosure in the present application, which may be combined with any other aspect unless specified otherwise, the first bone is selected from a group consisting of a metacarpal, a metatarsal, a tibia, a fibula, an ulna, a radius, and a phalange.

In another aspect of the disclosure in the present application, which may be combined with any other aspect unless specified otherwise, the second bone is selected from a group consisting of a metacarpal, a metatarsal, a tibia, a fibula, an ulna, a radius, and a phalange.

In another aspect of the disclosure in the present application, which may be combined with any other aspect unless specified otherwise, the first bone is a first metacarpal and the second bone is a second metacarpal.

In another aspect of the disclosure in the present application, which may be combined with any other aspect unless specified otherwise, the system is utilized in an ankle syndesmosis, an osteotomy, or a bunionectomy.

Accordingly, a need exists for a guided insertion system that can easily and accurately insert a cable through at least one bone.

Further, a need exists for a guided insertion system that maintains alignment and trajectory between two bones.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a side view of a button leader having a first leader end, a middle shaft, and a second leader end, according to one aspect of the present disclosure.

FIG. 2A is a perspective view of a lead button, according to one aspect of the present disclosure.

FIG. 2B is a top view of a lead button, according to one aspect of the present disclosure.

FIG. 2C is a left side view of a lead button, according to one aspect of the present disclosure.

FIG. 2D is a right side view of a lead button, according to one aspect of the present disclosure.

FIG. 2E is a side view of a lead button having an aperture that is off-center from an axis of the leading wing and trailing wing, according to one aspect of the present disclosure.

FIG. 3A illustrates a lead button interfacing with a first mating feature on a button leader, according to one aspect of the present disclosure.

FIG. 3B illustrates a lead button affixed to a cable interfacing with a button leader, according to one aspect of the present disclosure.

FIG. 4A illustrates an anchoring system with a cable affixed to the button leader, according to one aspect of the present disclosure.

FIG. 4B illustrates a button leader having a hole-shaped first mating feature, according to one aspect of the present disclosure.

FIG. 5A is a cross-sectional side view of a lead button interfacing with a first mating feature of a second leader end of a button leader, according to one aspect of the present disclosure.

FIG. 5B is a cross-sectional side view of a lead button interfacing with a first mating feature of a second leader end of a button leader, according to one aspect of the present disclosure.

FIG. 5C is a cross-sectional side view of a lead button interfacing with a cannulated button leader, according to one aspect of the present disclosure.

FIG. 5D is a cross-sectional side view of a lead button upon being decoupled from the button leader, according to one aspect of the present disclosure.

FIG. 6 is a cross-sectional side view of a button leader fully cinched across a first and second bone, according to one aspect of the present disclosure.

FIG. 7A illustrates a drill unit forming a channel through a first bone, according to one aspect of the present disclosure.

FIG. 7B illustrates a drill unit forming a first and second channel through a first and second bone, according to one aspect of the present disclosure.

FIG. 7C illustrates a drill unit forming a first and second channel through a first and second bone, according to one aspect of the present disclosure.

FIG. 7D illustrates a drill unit forming a first and second channel through a first and second bone, where the button leader has been removed from the button inserter, according to one aspect of the present disclosure.

FIG. 7E illustrates a button leader being pushed through a first and second bone, according to one aspect of the present disclosure.

FIG. 7F illustrates the button leader of FIG. 7E being further pushed through the first and second bones, according to one aspect of the present disclosure.

FIG. 7G illustrates the button leader of FIG. 7F being further pushed through the first and second bones, according to one aspect of the present disclosure.

FIG. 7H illustrates the button leader of FIG. 7G being further pushed through the first and second bones such that the cable is placed through the first and second channel, according to one aspect of the present disclosure.

FIG. 7I illustrates the button leader of FIG. 7H being cinched through the first and second bones, according to one aspect of the present disclosure.

FIG. 7J illustrates the button leader of FIG. 7I being further cinched through the first and second bones, according to one aspect of the present disclosure.

FIG. 7K illustrates the button leader of FIG. 7J being fully cinched through the first and second bones, according to one aspect of the present disclosure.

FIG. 8 is a flowchart outlining a method of inserting a cable through a first bone and a second bone, according to one aspect of the present disclosure.

FIG. 9 is a perspective view of a first spacing component of a trapezium spacer, according to one aspect of the present disclosure.

FIG. 10 is a perspective view of a second spacing component of a trapezium spacer, according to one aspect of the present disclosure.

FIG. 11 is a side view of a trapezium spacer inserted into a trapezial space by a Heiss Retractor, according to one aspect of the present disclosure.

### DETAILED DESCRIPTION

The present disclosure is directed to an anchoring system and a method for inserting a button leader and maintaining alignment through a first and second bone such that a lead button can be secured using a cable.

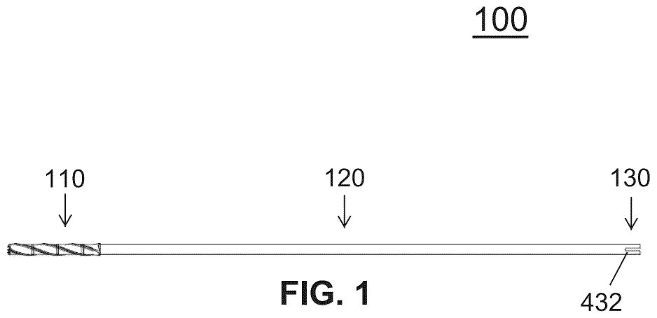

FIG. 1 illustrates an example button leader 100 according to a preferred embodiment of the present disclosure. The button leader 100 may include first leader end 110, a middle shaft 120, and a second leader end 130. In some embodiments, the first leader end 110 has a greater outer diameter than the middle shaft 120 and the second leader end 130. In other embodiments, the first leader end 110, middle shaft 120, and second leader end 130 have the same outer diameter.

The first leader end 110 may include a drill unit 140 designed to drill a hole or channel through a bone. In embodiments having a drill unit 140 such as FIG. 1, the drill unit 140 spans the entire first leader end 110. The drill unit 140 may include one or more flutes. The number of flutes shown in FIG. 1 is purely exemplary and other flute configurations may exist. In some examples, the drill unit 140 may include a sharpened tip 160 configured to pierce bone.

Preferably the middle shaft 120 is coupled to the first leader end 110 and is a cylindrical pole extending from the drill unit 140. The smooth surface of the middle shaft 120 may be configured to easily glide through the channel drilled into the bone by the drill unit 140. In some embodiments, the middle shaft 120 has a generally uniform outer diameter smaller than the outer diameter of the first leader end 110. In yet other embodiments, the diameter of the middle shaft 120 gradually decreases from the diameter of the first leader end 110 to the diameter of the second leader end 130. The diameter of the first leader end 110 may be approximately 0.091 to 2.3 millimeters (mm).

In other examples, the first leader end 110 may be connected to the second leader end 130 without a middle shaft 120 therebetween. In this embodiment, the change in diameter from the first leader end 110 to the second leader end 130 may be abrupt, and the portion between the first leader end 110 and the second leader end 130 may have a stepped configuration.

At the end opposite the first leader end 110, the middle shaft 120 is coupled with the second leader end 130 in a preferred embodiment. The second leader end 130 includes a first mating feature 432. The first mating feature 432 is preferably a slotted aperture allowing interaction with the lead button, for example a uniformed slot capable of receiving the lead button.

In other embodiments, the first mating feature may be an insertion tip, wedge, prong, or other specific geometry. The identified mating features in the application are exemplary, and other mating features may be added or omitted in other embodiments of the button leader 100.

The button leader 100 is merely an example of a button leader that may be used to deploy the lead button 300A. Any suitable button leader may be used to deploy the lead button 300A that is compatible with the advantages of the lead button 300A as described herein.

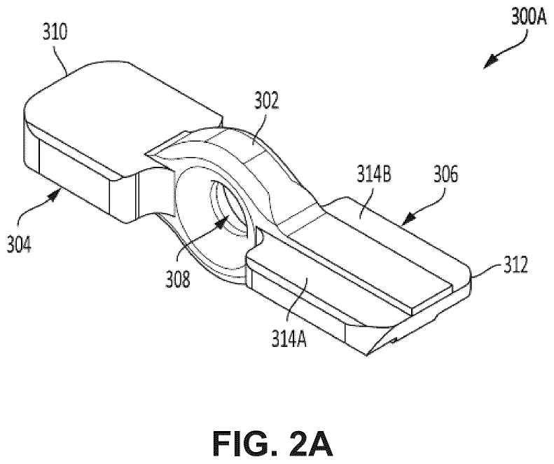

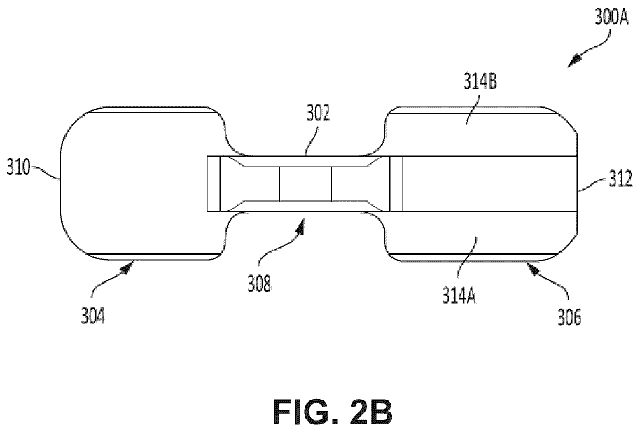

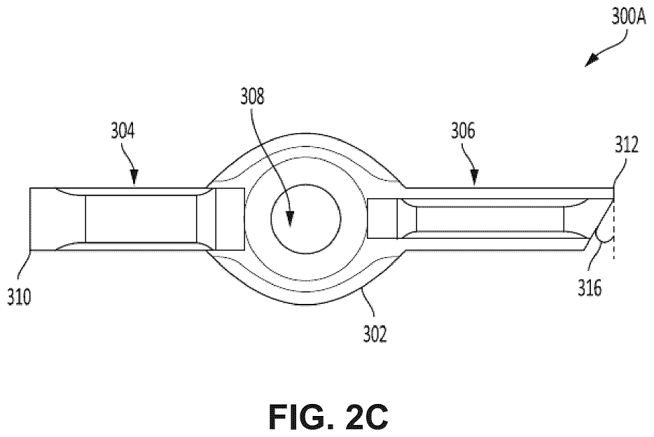

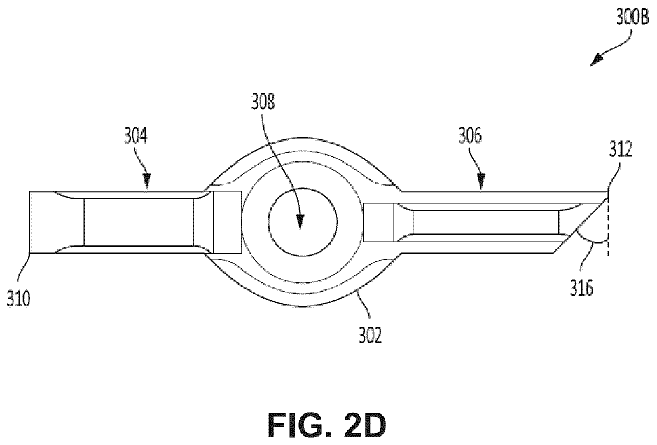

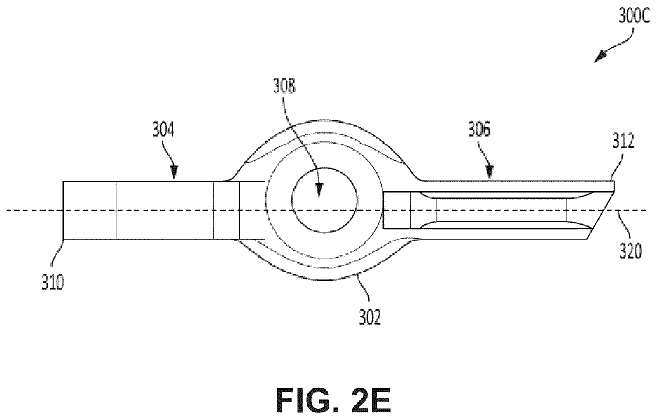

FIGS. 2A to 2D illustrate a perspective view, a top view, and side views respectively of an example lead button 300A. The lead button 300A preferably includes a support 302 having an aperture 308. The support 302 may include curved surfaces to reduce friction between cable and the support 302 during installation of the lead button 300A. In addition, the inclusion of a single aperture 308 in the support 302 for each cable strand to move through during installation of the lead button 300A may help reduce friction between the cable and the lead button 300A as compared to typical lead buttons having multiple apertures for different cable strands or different portions of a single cable strand.

The cable 418 is preferably a suture which may be a flexible material, e.g., cable or cable tape. In some instances, the cable 418 may be a single strand of cable. In other instances, the cable 418 may be multiple strands of cable that are arranged to extend between a first bone and a second bone. In at least one instance, the cable 418 can be an adjustable or non-adjustable loop. In at least one instance, the combination of the cable 418 and the lead button 300A can be an adjustable, knotless button/loop construct. The knotless button/loop construct may be self-locking.

In an alternative embodiment, rather than a button leader 100 and a button inserter 420 (two instruments), a combined guidewire portion at the tip (for example, approximately 2″ long), would provide the initial starting point and trajectory, while a stepped-up diameter further back with cutting geometry would provide an increased diameter hole to allow passage of the button. Behind this stepped-up cutting geometry, the diameter could then be stepped back down slightly to allow for decreased friction across the bones (or could remain the same diameter to aid with bony alignment). At the end could be a slot to engage the button (with pre-loaded cable) and/or button inserter. Or a cable loop could be swaged at the end to shuttle suture.

Referring again to the lead button 300A, leading wing 304 extends from the support 302 to a leading end 310 of the lead button 300A. A trailing wing 306 extends from the support 302 to a chamfered end 312 of the lead button 300A. The wings 304 and 306 may have various suitable lengths with respect to the support 302.

In various instances, the wing 306 may be configured to engage with a button leader tip such that the trailing wing 306 does not slide or otherwise move away from the button leader tip until the lead button 300A is deployed. For instance, the trailing wing 306 may include recesses 314A, 314B. The wing 306 may include the recesses 314A, 314B on a single side or on opposing sides (e.g., on the opposing side not illustrated). The non-recessed portion(s) of the trailing wing 306 may correspond to a recess or recesses in the button leader tip such that when the trailing wing 306 is slid within the button leader tip, lateral movement of the lead button (e.g., perpendicular to the long axis of the lead button 300A) is prevented with respect to the button leader.

The chamfered end 312 of the trailing wing 306 may include a chamfer at an angle 316. In various examples, the angle 316 may be equal to about thirty degrees. In other examples, the trailing wing 306 may be chamfered at another suitable angle 316, such as between fifteen and sixty degrees. For instance, FIG. 2D illustrates an example lead button 300B having a trailing wing 306 with a chamfered end 312 at an angle 316 of about forty-five degrees.

In various examples, such as those illustrated in FIGS. 2A to 2D, the support 302 and the aperture 308 through the support 302 may be centered relative to the leading wing 304 and trailing wing 306. In other examples, the support 302 and/or the aperture 308 may be off-center relative to the leading wing 304 and the trailing wing 306. For instance, FIG. 2E illustrates an example lead button 300C having a support 302 and an aperture 308 that is off-center from an axis 320 of the leading wing 304 and trailing wing 306. The support 302 and the aperture 308 may be off-center towards the longer side of the trailing wing 306 (e.g., due to the chamfered end 312) to help the lead button 300C flip into place during installation. In other examples, the support 302 may be centered while the aperture 308 is off-center. In some instances, the support 302 and the aperture 308 may be centered along the axis 320 such that the leading wing 304 and the trailing wing 306 have equal lengths. In other instances, the support 302 and/or the aperture 308 may be off-center along the axis 320. When the support 302 is off-center along the axis 320, either the leading wing 304 or the trailing wing 306 may have a longer length than the other.

The example lead buttons 300A, 300B, or 300C may be composed from any suitable medical-grade material capable of long-term contact with biological materials. For example, the lead buttons 300A, 300B, or 300C may be composed of nitinol.

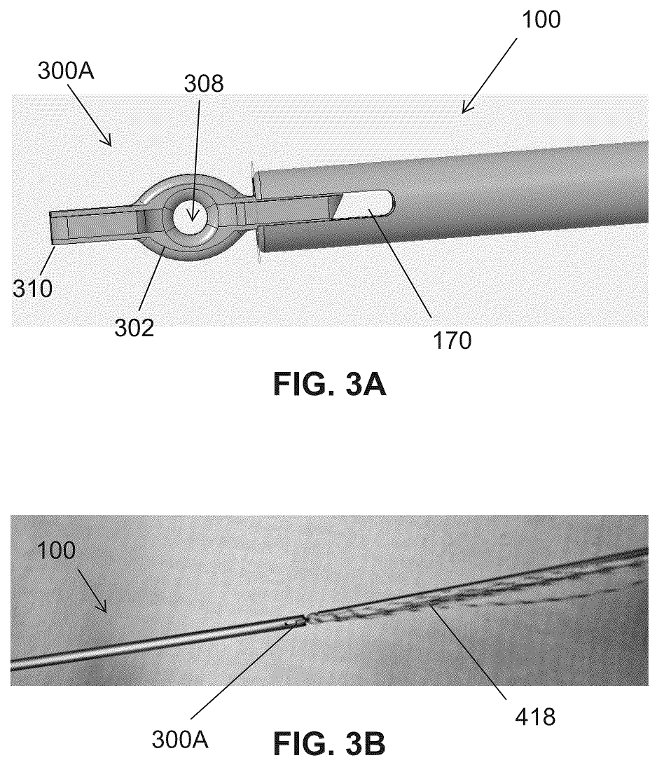

FIGS. 3A and 3B are views of the second leader end 130 interacting with the lead button 200. As shown in FIGS. 3A and 3B, the lead button 300A is captured within the first mating feature 432 of the second leader end 130 so that it is only configured to move along the long axis of the button leader. The lead button 300A may be held in place using pressure from a button inserter or the button may be held in place when the cable passed through the cannulated form of the button leader 100 to provide tension pulling towards the far bone.

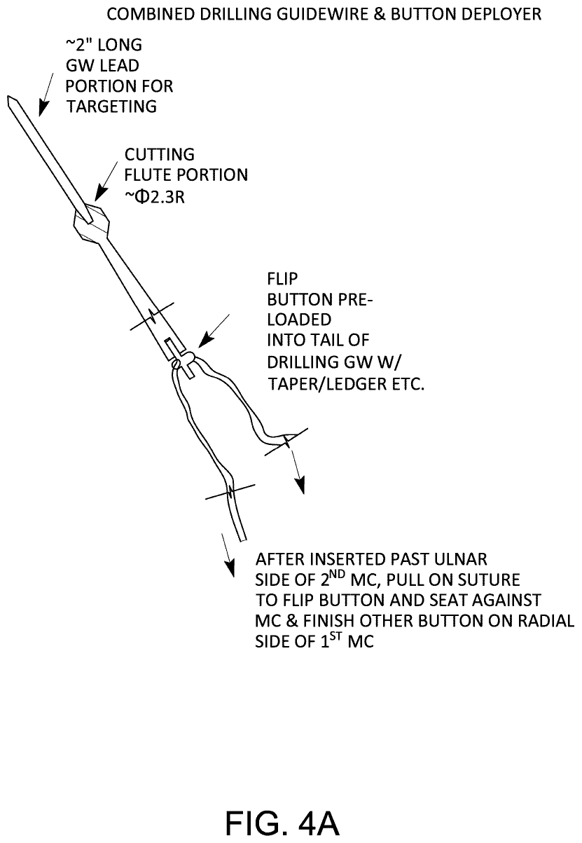

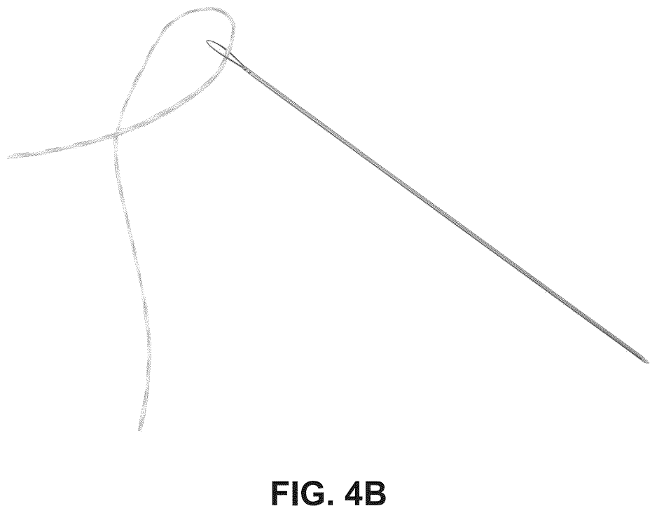

FIG. 4A illustrates an alternative anchoring system embodiment with a combined button inserter and button leader. When combined, once the button leader has been inserted into a second bone, the cable is pulled to secure the lead button, as described further below. FIG. 4B illustrates a button leader having a hole-shaped first mating feature.

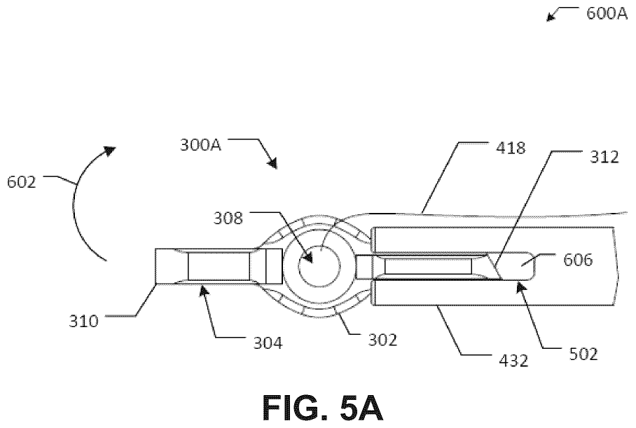

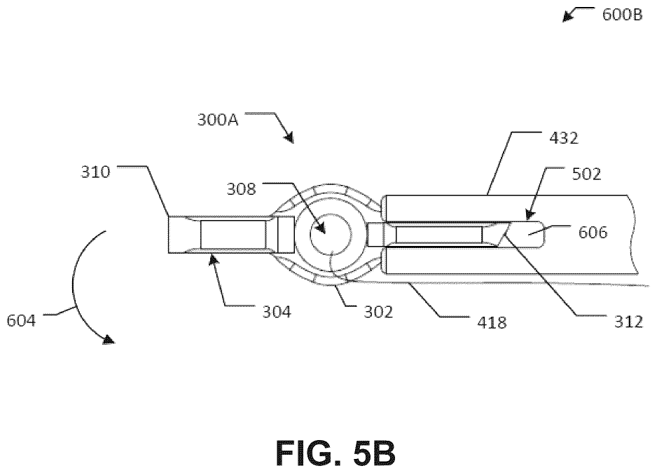

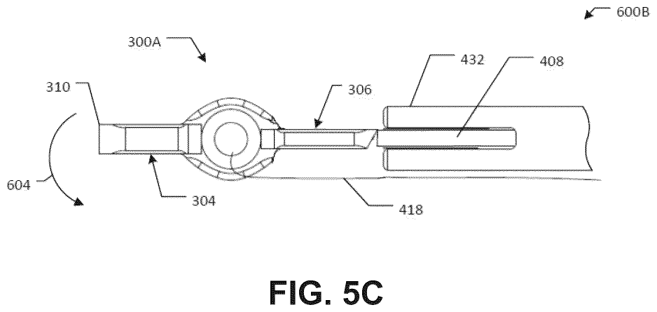

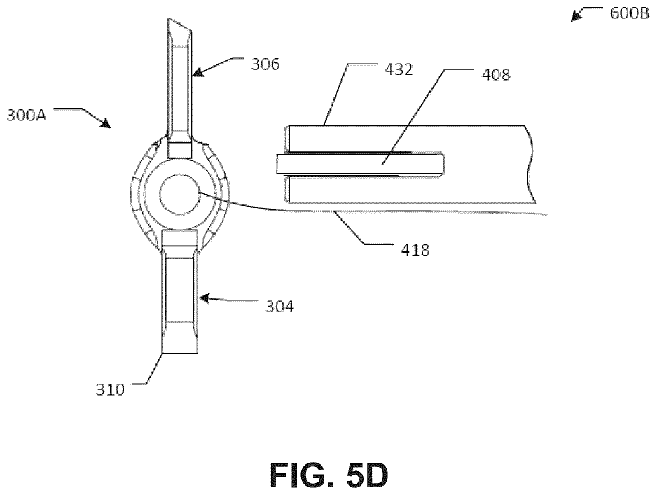

FIGS. 5A and 5B illustrate perspective side views of alternate configurations of the lead button 300A loaded within the first mating feature 432 of a button leader (e.g., the button leader 100). In the configuration 600A shown in FIG. 5A, the lead button 300A is positioned within the first mating feature 432 such that the chamfered end 312 is facing a first direction (e.g., towards the top of the page). In the configuration 600B shown in FIG. 5B, the lead button 300A is positioned within the first mating feature 432 such that the chamfered end 312 is facing opposite of the first direction (e.g., towards the bottom of the page). In each of the configurations 600A and 600B, the chamfered end 312 is facing towards the cable 418. The chamfered end 312 facing towards the cable 418 helps facilitate the lead button 300A flipping into place in a desired direction once the lead button 300A is deployed. For instance, in the configuration 600A, the lead button 300A flips in the direction of the arrow 602 upon deployment. Conversely, in the configuration 600B, the lead button 300A flips in the direction of the arrow 604 upon deployment.

In either the configuration 600A or 600B, the configuration of the lead button 300A enables the cable 418 to be to the side of the first mating feature 432 and button leader 100, rather than the cable 418 winding around the button leader shaft or around the lead button 300A itself In addition, the lead button 300A and the first mating feature 432 may be constructed such that a gap 606 remains between the lead button 300A and the first mating feature 432 when the lead button 300A is fully inserted. The gap 606 is radiolucent and is therefore visible under x-ray. The gap 606 may help a surgeon guide how far the button leader must be inserted before deploying the lead button 300A.

In some embodiments, the button leader 100 is cannulated such that the button leader 100 may be guided by a button inserter 420 having a trigger 422 which translates the pusher rod 408 to push the lead button 300A out of the first mating feature 432. In some embodiments, the button inserter 420 further comprises a locating window with the position of the locating window with respect to the at least one bone is identifiable on an x-ray.

FIGS. 5C and 5D further illustrate the configuration 600B to show an example of the lead button 300A flipping into place upon deployment. In various instances, the pusher rod 408 may extend beyond the end of the first mating feature 432 when the trigger is fully depressed, which may help ensure that the lead button 300A is fully separated from the button leader 100 and pushed all the way out of the first mating feature 432. Ensuring that the lead button 300A is fully separated from the button leader 100 may allow for easier deployment when installation of the lead button 300A involves pushing against resisting tissue (e.g., skin). In addition, the second leader end 408 that contacts the lead button 300A may be flat, as illustrated, such that the second leader end 408 only contacts the nearest portion of the chamfered end 312. The space between the flat end of the pusher rod 408 and the chamfered end 312 of the lead button 300A allows space for the lead button 300A to rotate or flip.

Upon the lead button 300A being pushed all the way out of the first mating feature 432, tension in the cable 418 causes the lead button 300A to flip to the side of the cable 418 (e.g., in the direction of the arrow 604). The chamfered surface 312 of the lead button 300A and the minimal contact between the pusher rod 408 and the chamfered surface 312 help facilitate the lead button 300A flipping towards the side of the cable 418. Facilitating the lead button 300A flipping in a desired or target direction may help reduce complications during a surgical procedure that may arise by the lead button 300A flipping in an undesired direction, which may potentially cause the cable 418 to tangle or get pinched. The provided flipping facilitation of the present disclosure also enables the lead button 300A to flip very close to the first mating feature 432, which can increase the ease of deploying the lead button 300A. FIG. 5D shows the flipped lead button 300A.

As indicated, the lead button 300A may be utilized as part of the cable-button technique to secure two bones together. For example, a method of ankle syndesmosis repair (with or without ankle fracture) may include drilling a bone hole through a patient's fibula and tibia. A button leader (e.g., the button leader 400) may be loaded with the lead button 300A and the lead button 100A. A surgeon may transport the lead button 300A through the bone hole via the button leader.

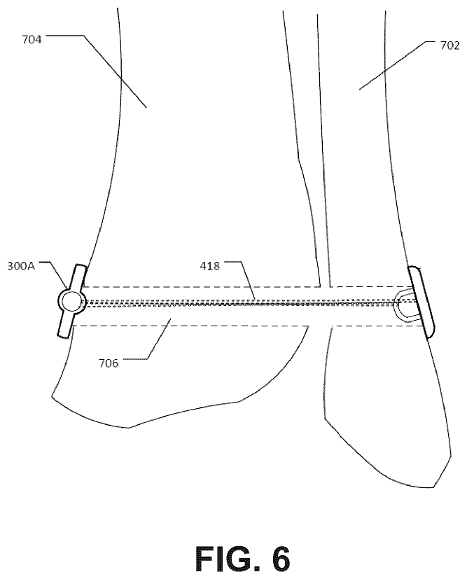

FIG. 6 illustrates the lead button 300A transported through a bone hole 706 in a fibula bone 702 and a tibia bone 704 via the button leader 400. Cable 418 couples the lead button 300A to the lead button 100A. A surgeon may then deploy the lead button 300A and the lead button 100A, such as by activating the trigger 406 on the button leader 400 and then translating the button leader 400 away from the patient. In some instances, the lead button 300A may deploy first, and then the lead button 100A may be deployed once the lead button 300A is in position. The pulley peg of the lead button 300A is fully within the bone hole 706, which enables solely the button head 102 of the lead button 300A to protrude from the surface of the fibula bone 702. In some instances, a knot of cable 418 may be positioned within the aperture 106 of the lead button 300A. In other embodiments, the button leader 100 may take a non-cannulated form using a solid tip button and function in an identical way using a feature that mates with a second button at the near end.

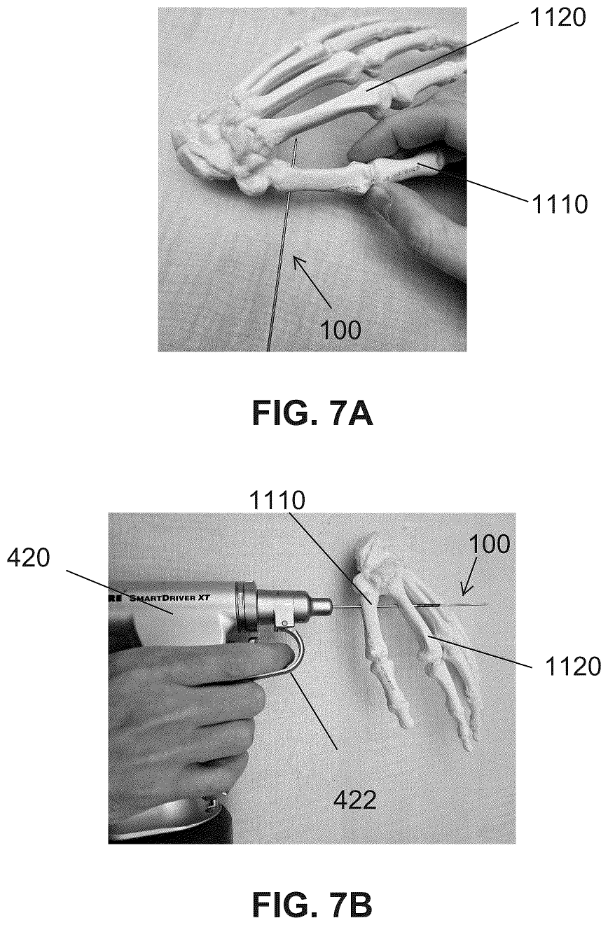

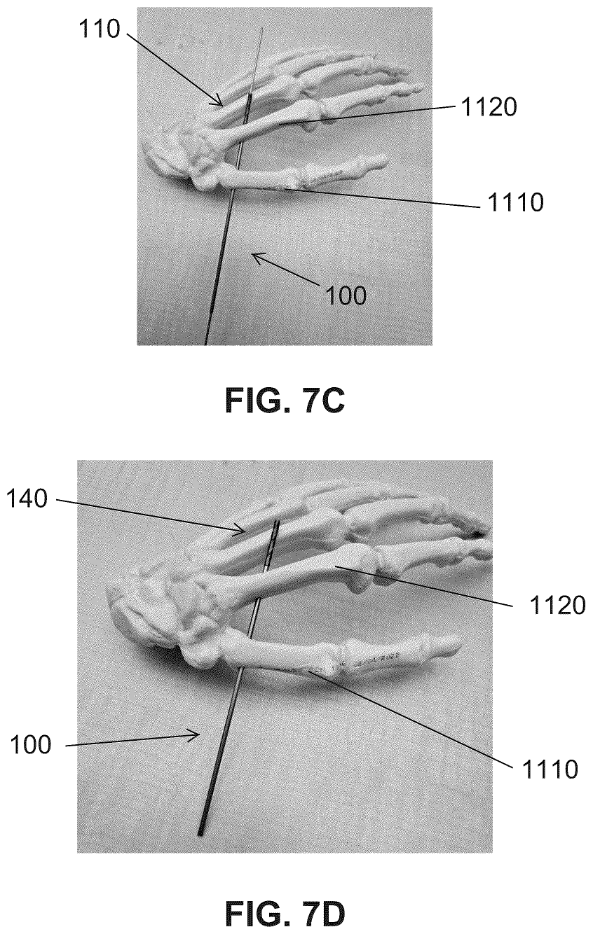

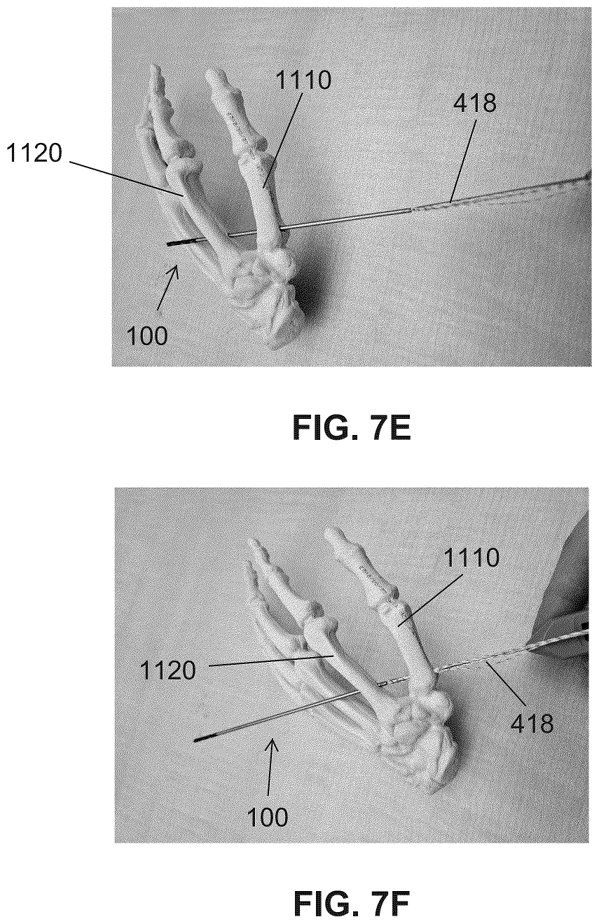

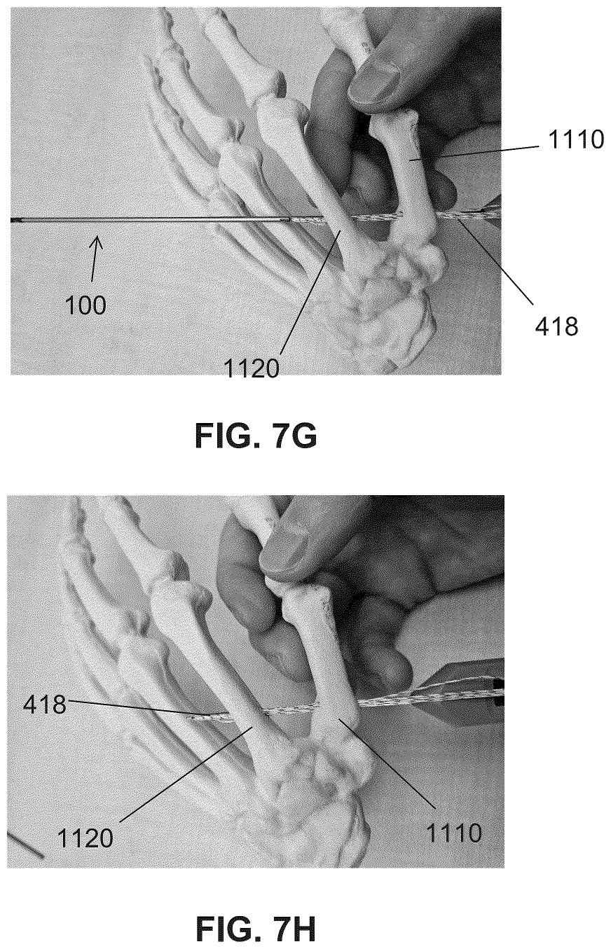

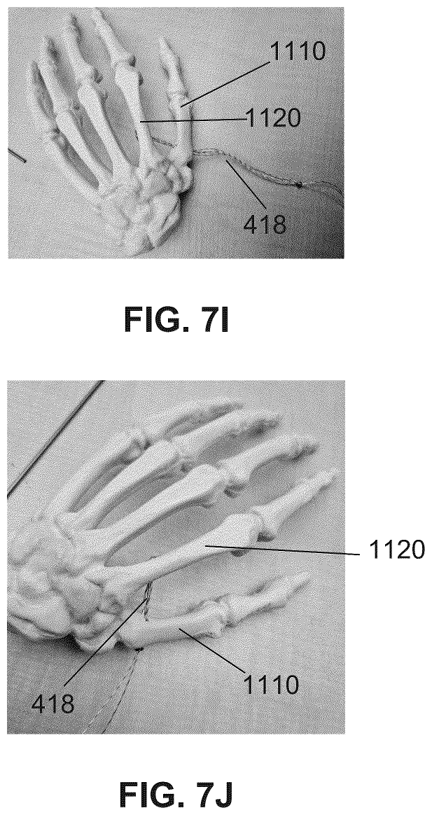

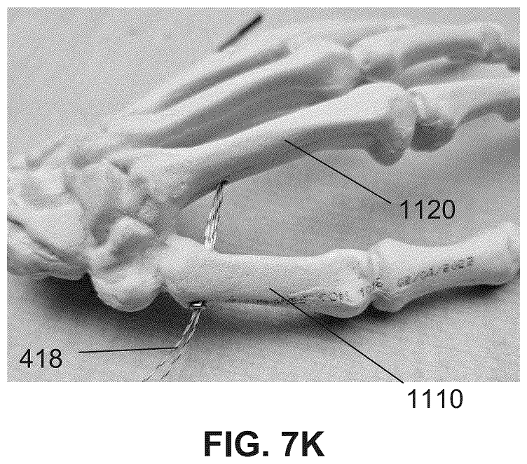

FIG. 7A illustrates a button leader 100 being inserted into a first bone 1110. While the bone may be any bone, FIG. 7A includes a first and second bone as a first and second metacarpal, respectively. Other such bone examples include, but are not limited to, a metatarsal, a tibia, a fibula, an ulna, a radius, and a phalange. During a surgery, a surgeon would use the drill unit 140 to drill a hole through the first bone 1110, stopping prior to entering the second bone 1120. At this time, the surgeon may adjust the first bone 110, hold it at the desired length and angle it while confirming the adjustment under fluoroscopy. When the orientation is satisfactory, the surgeon continues to advance the button leader 100 through the second bone 1120 as shown in FIG. 7B.

In some embodiments, a wire driver is used to hold the 2.3 mm cannulated button leader. The first leader end 110 of the button leader 100 is advanced until the drill tip 140 is completely through both metacarpals as illustrated in FIGS. 7C and 7D. As shown in FIG. 7D, from there a surgeon's grip on the button leader 100 is pulled out of the button inserter, and the button leader 100 is left in place across the first and second bones 1110, 1120. The advancing of the button leader 100 should slide smoothly across the channels due to the middle shaft 120 having a smaller diameter than the first leader end 110.

An additional advantage to this method is that by keeping the button leader and pulling the cable from the hole, there is no risk in losing all instrumentation that marks the hole axis. Traditionally, button leaders are removed and the cables are left in place to guide implants, however in that scenario there is a significant risk of the cable coming out with the button leader, resulting in a loss of targeting. The cable must then be replaced within the hole resulting in additional steps and frustration if the location cannot be easily found.

Once the drilling of the first and second channels is complete, a lead button is then secured to the second leader end 140 of the button leader 100. As shown in FIGS. 7E to 7H, the button leader is pushed forward until the cable is fully through both the first and second channel. During this process, the surgeon should not back up the button leader (i.e., moving in a first from the second bone to the first bone) as it could disconnect the lead button 300A from the back of the second leader end 130. The button leader 100 is pushed so that the lead button 300A and button leader 100 connection is pushed through the first and second channels under a button opposite the lead button 300A emerges from the far side of the second bone. At this point, the button leader 100 typically falls from the lead button 300A.

The trigger of the button inserter can deploy the flip button of the lead button 300A. A surgeon may pull backwards along the long axis of the button leader 100 or may manually release the lead button 300A. The cable 418 is then cinched by pulling backwards along the long axis of the button leader 100. Proper cinching is often cinching slowly until the desired fixation is achieved.

When the fixation is achieved, the surgeon cuts the only remaining cable flesh with the top of another button opposite the lead button 300A as shown by FIGS. 7I to 7K. Care should be taken not to further cinch while cutting this cable.

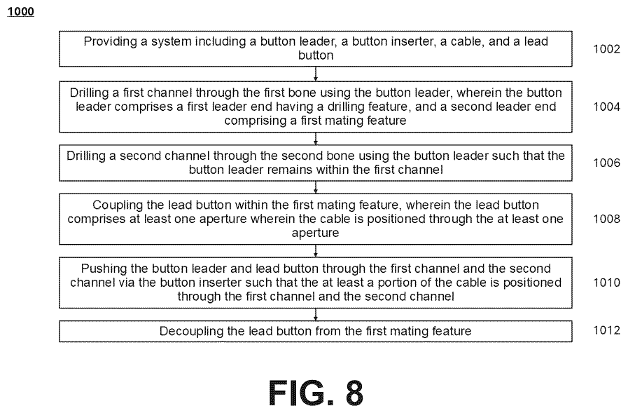

FIG. 8 illustrates a method of inserting a cable through a first bone and a second bone 1000. In Step 1002, a system is provided including a button leader, a button inserter, the cable, and a lead button. In Step 1004, a first channel is drilled through the first bone using the button leader which comprises a first leader end having a drilling feature, and a second leader end comprising a first mating feature. In Step 1006, a second channel is drilled through the second bone using the button leader such that the button leader remains within the first channel. In Step 1008, the lead button is coupled within the first mating feature. The lead button comprises at least one aperture wherein the cable is positioned through the at least one aperture. In Step 1010, the button leader is pushed and lead button through the first channel and the second channel via the button inserter such that the at least a portion of the cable is positioned through the first channel and the second channel. In Step 1012, the lead button is decoupled from the first mating feature.

A surgeon may use both the provided lead button and the provided lead button when performing the lead button technique, such as in an ankle syndesmosis, an osteotomy, a trapeziectomy, or a bunionectomy procedure. Alternatively, a surgeon may use the provided lead button with another suitable lead button or anchor, or may use the provided lead button with another suitable lead button or anchor.

After a trapeziectomy procedure, a trapezium spacer may be necessary to minimize over-compression of the first (thumb) metacarpal and second (index) metacarpal while the method of FIG. 8 is utilized. As a patient's trapezium bone is removed during the procedure, a trapezium spacer is placed into a trapezial space after trapeziectomy has been performed. As described further below, the user places a trapezium spacer including two spacing components between the base of a patient's first and second metacarpal and the scaphoid, ensuring it is providing space between the bones. Then, a placing instrument is expanded to enable distraction on the thumb and maintain proper alignment of the first and second metacarpal while the method of FIG. 8 is implemented. The user distracts the thumb to length via the trapezium spacer by ensuring the thumb should not be in a subsided position or an over-distracted position. In some embodiments, the trapezium spacer may be combined with other instruments such as a drill guide.

An example trapezium spacer includes two spacing components, a first spacing component configured to interact with the first metacarpal and a second spacing component configured to interact with the scaphoid. In some embodiments, the first and second spacing components are physically separated and in other embodiments, the first and second spacing components may be directly or indirectly connected. In yet other embodiments, the first spacing component is utilized without the second spacing component.

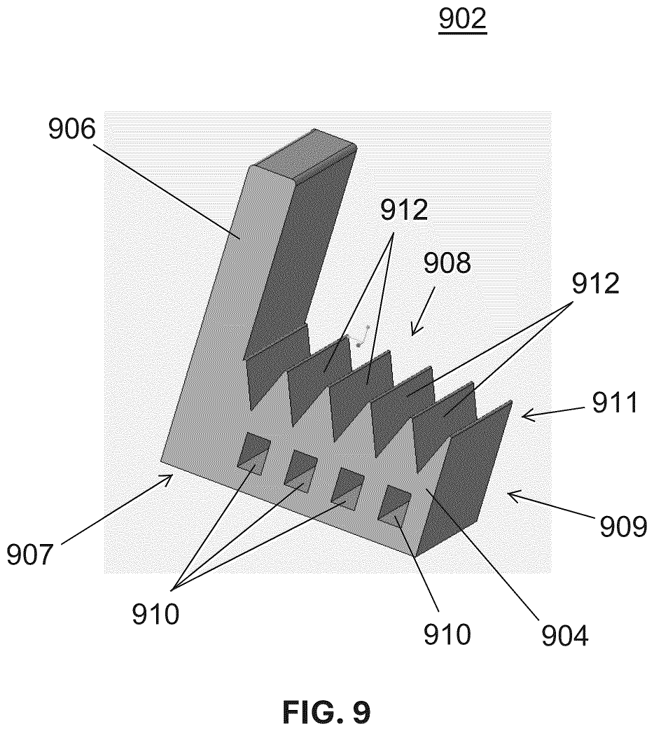

FIG. 9 illustrates an example first spacing component 902 according to an example of the present disclosure. The first spacing component 902 includes a body 904 and a flange 906. The body 904 of the first spacing component 902 is generally rectangular and includes a front face 907, a left side face (not pictured), a right side face 909, a back face (not pictured), a top face 911, and a bottom face (not pictured). The body 904 of the first spacing component 902 further includes a gripping surface 908 and guide holes 910.

In some embodiments, the body 904 of the first spacing component 902 may be made of or include a rigid material, such as hard plastics, metals, or any combinations thereof. For example, the body 904 of the first spacing component 902 may be made of or include a suitable metal (e.g., cobalt, notinol (nickel titanium), stainless steel) and/or a suitable plastic (e.g., polyethylenes, polyetheretherketones (PEEK), polylactic acid copolymers, polyglycolic copolymers).

In other embodiments, the body 904 of the first spacing component 902 may be made of or include a soft material like a textile. The textile material can include polyester, polytetrafluoroethylene (PTFE), expanded polytetrafluoroethylene (ePTFE), polyethylene terephthalate (PET) or any other suitable biocompatible-grade material or a combination thereof. In some examples, the body 904 of the first spacing component 902 may be made of or include a biological material. The biological material can include collagen, (allograft) tendon, muscle, fat, skin, or any other suitable joint interposition material or a combination thereof. In some examples, the body 904 of the first spacing component 902 may be made out of any other suitable implantable materials, such as polyurethane urea, silicone, and pyrocarbon. The body 904 of the first spacing component 902 may be made out of combinations of the materials as described herein. In some embodiments, the body 904 of the first spacing component 902 may be made of a (woven) suture.

In some embodiments, the body 904 of the first spacing component 902 may be one-size fits all for each patient. In other embodiments, the body 904 of the first spacing component 902 may be a custom size based on a patient's anatomy, such as the size of and the space between the first and second metacarpals. A user can determine the anatomy of the patient by any suitable means, such as by x-ray.

In some embodiments, the body 904 of the first spacing component 902 may have a length in the range of about 1 mm to about 10 mm, for example, about 1 mm to about 3 mm, about 3 mm to about 5 mm, about 5 mm to about 7 mm, or about 7 mm to about 10 mm. In other embodiments, the body 904 of the first spacing component 902 may have any other suitable length. In some embodiments, the body 904 of the first spacing component 902 may have a width less than or equal to the length of the body 904.

The top face 911 of the body 904 includes a gripping surface 908. As shown in FIG. 9, the gripping surface 908 of the body 904 includes a plurality of teeth 912 configured to interact with the first metacarpal and increase friction between the first metacarpal and the first spacing component 902. The teeth 912 may be pointed or rounded at the apex.

In some embodiments such as FIG. 9, there are five teeth 912. In other embodiments, there may be four teeth. The number of teeth on the first spacing component may differ in other embodiments. The number of teeth may depend on the size of the first metacarpal, the size of the instrument utilized to place the first spacing component in the trapezial space, the patient's anatomy, or the amount of friction necessary to ensure the first spacing component remains stationary to maintain alignment of the bones. The use of five teeth in the description below is purely exemplary.

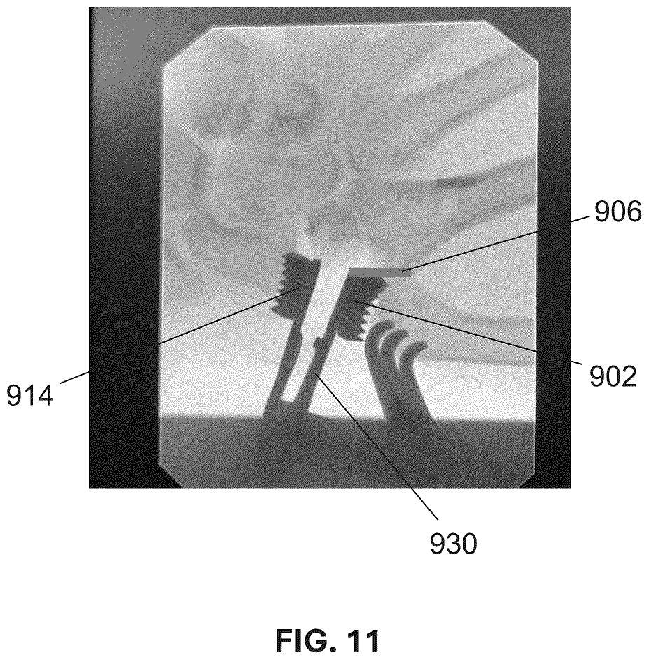

In some embodiments the gripping surface 908 may include a curvature that is concave with respect to the body 904 as shown in FIG. 11. By raising the height of teeth towards the ends of the gripping surface 908, the gripping surface 908 may be better suited to interact with the natural convex curvature of the first metacarpal.

The identified gripping surfaces are exemplary and other gripping surfaces may be added or omitted in other embodiments of the first spacing component 902. In other embodiments, the gripping surface texture and size may vary. Some examples of other gripping surfaces include, but are not limited to, a knurled surface, an anatomic trapezial-shaped surface, or a gripping surface intended to mirror the base of the first metacarpal.

The body 904 of the first spacing component 902 includes guide holes 910 that extend through the front face 907 and a back face (not pictured). The guide holes 910 are capable of interacting with a placing instrument as described further below configured to assist with the insertion and removal of the trapezium spacer from a patient's trapezial space.

The guide holes 910 may vary in size and shape based on the type of placing instrument used. In the illustrated embodiments, the guide holes 910 are rectangular with respect to the front face 907. In other embodiments, the guide holes may be square, circular, or any other shape capable of interacting with the placing instrument. The width of the guide holes may vary from the front face to the back face in other embodiments.

In some embodiments such as FIG. 9, there are four guide holes 910. In other embodiments, there may be fewer guide holes. The number of guide holes on the first spacing component may differ in other embodiments. The number of guide holes may depend on the type of placing instrument, the size of the placing instrument, and the shape and size of the body of the first spacing component. In other embodiments, no guide holes are used and a placing instrument interacts with the first spacing component differently. The use of four guide holes in the description is purely exemplary.

The first spacing component 902 is configured to provide interposition and maintain space between the first and second metacarpal through a flange 906. The flange 906 extends from the left side face (not pictured) and the top face 911 adjacent to the gripping surface 908.

In some embodiments, the flange 906 of the first spacing component 902 may be made of or include a rigid material, such as hard plastics, metals, or any combinations thereof. For example, the flange 906 of the first spacing component 902 may be made of or include a suitable metal (e.g., cobalt, notinol (nickel titanium), stainless steel) and/or a suitable plastic (e.g., polyethylenes, polyetheretherketones (PEEK), polylactic acid copolymers, polyglycolic copolymers).

In other embodiments, the flange 906 of the first spacing component 902 may be made of or include a soft material like a textile. The textile material can include polyester, polytetrafluoroethylene (PTFE), expanded polytetrafluoroethylene (ePTFE), polyethylene terephthalate (PET) or any other suitable biocompatible-grade material or a combination thereof. In some examples, the flange 906 of the first spacing component 902 may be made of or include a biological material. The biological material can include collagen, (allograft) tendon, muscle, fat, skin, or any other suitable joint interposition material or a combination thereof. In some examples, the flange 906 of the first spacing component 902 may be made out of any other suitable implantable materials, such as polyurethane urea, silicone, and pyrocarbon. The flange 906 of the first spacing component 902 may be made out of combinations of the materials as described herein. In some embodiments, the flange 906 of the first spacing component 902 may be made of a (woven) suture. In some embodiments, the material of the flange 906 and the material of the body 904 of the first spacing component 902.

In some embodiments, the flange 906 of the first spacing component 902 may be one-size fits all for each patient. In other embodiments, the flange 906 of the first spacing component 902 may be a custom size based on a patient's anatomy, such as the size of and the space between the first and second metacarpals. A user can determine the anatomy of the patient by any suitable means, such as by x-ray.

In some embodiments, the flange 906 of the first spacing component 902 may have a length in the range of about 1 mm to about 10 mm, for example, about 1 mm to about 3 mm, about 3 mm to about 5 mm, about 5 mm to about 7 mm, or about 7 mm to about 10 mm. In other embodiments, the flange 906 of the first spacing component 902 may have any other suitable length. In some embodiments, the flange 906 of the first spacing component 902 may have a width less than or equal to the length of the flange 906.

The identified flange is exemplary and other flanges may be added or omitted in other embodiments of the first spacing component 902. In other embodiments, the flange shape and size may vary. Some examples of differing flange shapes include, but are not limited to, a rectangle with a chamfered end (illustrated in FIG. 9), a flat rectangle, a rectangle with a rounded end, a tapered rectangle, an anatomic joint surface, and a forked body.

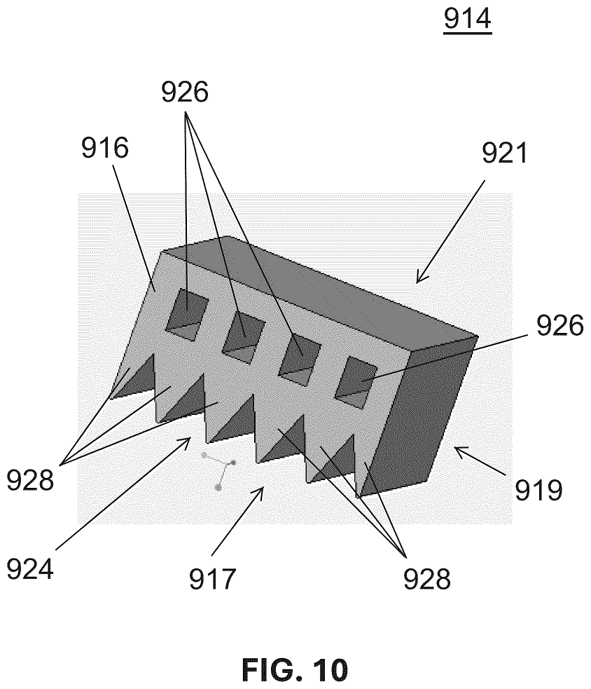

FIG. 10 illustrates an example second spacing component 914 configured to maintain the trapezial gap according to an example of the present disclosure. The second spacing component 914 includes a body 916. The body 916 of the second spacing component 914 is generally rectangular and includes a front face 917, a left side face (not pictured), a right side face 919, a back face (not pictured), a top face 921, and a bottom face (not pictured). The body 916 of the second spacing component 914 further includes a gripping surface 924 and guide holes 926. The top face 921 of the second spacing component 914 is opposite the bottom face of the first spacing component 902 when in use.

In some embodiments, the body 916 of the second spacing component 914 may be made of or include a rigid material, such as hard plastics, metals, or any combinations thereof. For example, the body 916 of the second spacing component 914 may be made of or include a suitable metal (e.g., cobalt, notinol (nickel titanium), stainless steel) and/or a suitable plastic (e.g., polyethylenes, polyetheretherketones (PEEK), polylactic acid copolymers, polyglycolic copolymers).

In other embodiments, the body 916 of the second spacing component 914 may be made of or include a soft material like a textile. The textile material can include polyester, polytetrafluoroethylene (PTFE), expanded polytetrafluoroethylene (ePTFE), polyethylene terephthalate (PET) or any other suitable biocompatible-grade material or a combination thereof. In some examples, the body 916 of the second spacing component 914 may be made of or include a biological material. The biological material can include collagen, (allograft) tendon, muscle, fat, skin, or any other suitable joint interposition material or a combination thereof. In some examples, the body 916 of the second spacing component 914 may be made out of any other suitable implantable materials, such as polyurethane urea, silicone, and pyrocarbon. The body 916 of the second spacing component 914 may be made out of combinations of the materials as described herein. In some embodiments, the body 916 of the second spacing component 914 may be made of a (woven) suture. In some embodiments, the material of the second spacing component 914 and the material of the body 904 of the first spacing component 902.

In some embodiments, the body 916 of the second spacing component 914 may be one-size fits all for each patient. In other embodiments, the body 916 of the second spacing component 914 may be a custom size based on a patient's anatomy, such as the size of and the space between the first and second metacarpals. A user can determine the anatomy of the patient by any suitable means, such as by x-ray.

In some embodiments, the body 916 of the second spacing component 914 may have a length in the range of about 1 mm to about 10 mm, for example, about 1 mm to about 3 mm, about 3 mm to about 5 mm, about 5 mm to about 7 mm, or about 7 mm to about 10 mm. In other embodiments, the body 916 of the second spacing component 914 may have any other suitable length. In some embodiments, the body 916 of the second spacing component 914 may have a width less than or equal to the length of the body 916.

The bottom face 921 of the body 916 includes a gripping surface 924. Unlike the first spacing component 902, the gripping surface 924 of the second spacing component 914 extends along the entire bottom face of the second spacing component 914. As shown in FIG. 10, the gripping surface 924 of the body 916 includes a plurality of teeth 928 configured to interact with the first metacarpal and increase friction between the scaphoid and the second spacing component 914. The teeth 928 may be pointed or rounded at the apex.

In some embodiments such as FIG. 10, there are six teeth 928. In other embodiments, there may be five teeth. The number of teeth on the second spacing component may differ in other embodiments. The number of teeth may depend on the size of the scaphoid, the size of the instrument utilized to place the second spacing component in the trapezial space, the patient's anatomy, or the amount of friction necessary to ensure the second spacing component remains stationary to maintain alignment of the bones. The use of six teeth in the description below is purely exemplary.

In some embodiments the gripping surface 924 may include a curvature that is concave with respect to the body 916 as shown in FIG. 11. By raising the height of teeth towards the ends of the gripping surface 924, the gripping surface 924 may be better suited to interact with the natural convex curvature of the scaphoid.

The identified gripping surfaces are exemplary and other gripping surfaces may be added or omitted in other embodiments of the second spacing component 914. In other embodiments, the gripping surface texture and size may vary. Some examples of other gripping surfaces include, but are not limited to, a knurled surface, an anatomic trapezial-shaped surface, or a gripping surface intended to mirror the base of the scaphoid. The gripping surface texture and size of the second spacing component may be similar or different from the first spacing component.

The body 916 of the second spacing component 914 includes guide holes 926 that extend through the front face 917 and a back face (not pictured). The guide holes 926 are capable of interacting with a placing instrument as described further below configured to assist with the insertion and removal of the trapezium spacer from a patient's trapezial space.

The guide holes 926 may vary in size and shape based on the type of placing instrument used. In the illustrated embodiments, the guide holes 926 are rectangular with respect to the front face 917. In other embodiments, the guide holes may be square, circular, or any other shape capable of interacting with the placing instrument. The width of the guide holes may vary from the front face to the back face in other embodiments.

In some embodiments such as FIG. 10, there are four guide holes 926. In other embodiments, there may be fewer guide holes. The number of guide holes on the second spacing component may differ in other embodiments. The number of guide holes may depend on the type of placing instrument, the size of the placing instrument, and the shape and size of the body of the second spacing component. In other embodiments, no guide holes are used and a placing instrument interacts with the second spacing component differently. The use of four guide holes in the description is purely exemplary.

In some embodiments, no second spacing component is utilized. In embodiments where only the first spacing component is utilized, the first spacing component includes an additional gripping surface on the bottom face.

FIG. 11 is an x-ray image depicting the placing instrument 930 holding the thumb to length in the trapezial space. In FIG. 11, the first spacing component 902 and the second spacing component 914 being inserted into a patient's trapezial space after a trapeziectomy procedure by a placing instrument 930. The placing instrument 930 provides the function of temporarily holding the thumb to length while inside the trapezial space, and providing a means to prevent over-compression of the first and second metacarpals via the flange 906. The placing instrument may be any instrument suitable to insert the first spacing component 902 and the second spacing component 914 into a patient's trapezial space. One example of a placing instrument is a Heiss Retractor, though other instruments including standard retractors, spreaders, clamps, and forceps may be utilized with the first spacing component. The placing instrument-a Heiss Retractor-may include extending pieces, such as spikes, designed to be inserted into the guide holes of the first spacing component 902 and the second spacing component 914.

In embodiments where the placing instrument is a Heiss Retractor, the placing instrument, already interacting with the first spacing component and the second spacing component, is placed into the trapezial space after trapeziectomy has been performed. The user must first place the flange 906 of the first spacing component 902 between the base of the first and second metacarpal, ensuring the flange 906 is providing space between the bones. Then, the placing instrument 930 is expanded to provide distraction on the thumb. The user ensures that the thumb is distracted to length, the thumb should not be in a subsided position or an over-distracted position. The user leaves the placing instrument while installing a suture suspensionplasty implant construct as described above in FIG. 8. The flange 906 maintains distance between the first and second metacarpals while the suture system is tightened, preventing over-compression between the two bones. Once the implant construct is secured, the placing instrument 930, the first spacing component 902, and the second spacing component 914 are removed, allowing natural motion between the bones. The examples and embodiments disclosed herein are to be construed as merely illustrative and not a limitation of the scope of the present disclosure in any way. It will be apparent to those having skill in the art that changes may be made to the details of the above described embodiments without departing from the underlying principles discussed. In other words, various modifications and improvements of the embodiments specifically disclosed in the description above are within the scope of the appended claims. For example, any suitable combination of features of the various embodiments described is contemplated.

What is claimed is:

1. A system for transporting an anchoring system through at least one bone, comprising:
a button leader comprising:
a first leader end and a second leader end, and
a first mating feature formed on the second leader end; a cable; and
a lead button comprising:
at least one aperture, wherein the cable is positioned through the aperture, a leading wing extending from the aperture, and
a trailing wing extending from the aperture opposite the leading wing, wherein the leading wing is operable to mate with the first mating feature,
wherein the button leader and the lead button are configured to be transported together through a channel of at least one bone.

2. The system of claim 1, wherein a bone drilling feature is formed on the first leader end.

3. The system of claim 1, wherein a diameter of the first leader end is greater than a diameter of the second leader end.

4. The system of claim 1, wherein a diameter of the first leader end is in a range of about 1.2 mm to about 6.5 mm.

5. The system of claim 1, wherein the button leader is cannulated.

6. The system of claim 1, further comprising a button inserter, the button inserter being connected to the lead button, the button inserter further comprises, a second mating feature operable to mate with the trailing wing and a pusher rod, wherein the pusher rod is operable to decouple the lead button from the button inserter.

7. The system of claim 6, further comprising a trigger, wherein the trigger is operable to cause the pusher rod to translate within the button inserter.

8. The system of claim 6, wherein the button inserter further comprises a locating window, wherein the position of the locating window with respect to the at least one bone is identifiable on an x-ray.

9. The system of claim 6, wherein the button inserter and the button leader are coupled.

10. The system of claim 1, further comprising a fixator that is coupled to the lead button by the cable, wherein the fixator is an anchor or an additional button.

11. The system of claim 1, wherein the trailing wing includes a chamfered end.

12. The system of claim 11, wherein the chamfered end is chamfered at an angle between 20 to 60 degrees relative to the trailing wing.

13. The system of claim 1, wherein the aperture is formed off-center from a midpoint between the leading wing and the trailing wing.

14. The system of claim 1, wherein the at least one bone is selected from a group consisting of a metacarpal, a metatarsal, a tibia, a fibula, an ulna, a radius, and a phalange.

15. The system of claim 1, wherein the button is configured to passed through channels in multiple bones.

16. The system of claim 1, wherein one bone is a first metacarpal and a second bone is a second metacarpal.

17. A method of inserting a cable through a first bone and a second bone, the method comprising:
providing a system including a button leader, a button inserter, the cable, and a lead button;
drilling a first channel through the first bone using the button leader, wherein the button leader comprises a first leader end having a drilling feature, and a second leader end comprising a first mating feature;
drilling a second channel through the second bone using the button leader such that the button leader remains within the first channel;
coupling the lead button within the first mating feature, wherein the lead button comprises at least one aperture wherein the cable is positioned through the at least one aperture;
pushing the button leader and lead button through the first channel and the second channel via the button inserter such that the at least a portion of the cable is positioned through the first channel and the second channel; and
decoupling the lead button from the first mating feature.

18. The method of claim 17, wherein the lead button further includes:
a leading wing extending from the aperture; and
a trailing wing extending from the aperture,
wherein the leading wing is operable to mate with the first mating feature and the trailing wing is operable to mate with a second mating feature, the second mating feature formed in a first end of a button inserter.

19. The method of claim 18, wherein the trailing wing includes a chamfered end.

20. The method of claim 19, wherein the chamfered end is chamfered at an angle between 20 to 60 degrees relative to the trailing wing.
